(19) United States
(12) Patent Application Publication
(10) Pub. No.: US 2026/0275393 A1
(43) Pub. Date: Sep. 17, 2026

(54) METHODS TO IMPROVE BIOPRODUCT PRODUCTION IN CYANOBACTERIA

(71) Applicant: MORGAN STATE UNIVERSITY, Baltimore, MD (US)

(72) Inventors: Viji Sitther, Baltimore, MD (US); Mst Sayadujjhara, Parkville, MD (US); Yavuz Yalcin, Baltimore, MD (US)

(21) Appl. No.: 19/554,058

(22) Filed: Mar. 2, 2026

Related U.S. Application Data
(60) Provisional application No. 63/764,636, filed on Feb. 28, 2025.
(60) Provisional application No. 63/810,583, filed on May 22, 2025.

(51) Int. Cl. C12P 7/6409; B82Y 5/00; C12N 1/205; C12N 1/38; C12N 13/00; C12R 1/01

(52) U.S. Cl. CPC C12P 7/6409; C12N 1/205; C12N 1/38; C12N 13/00; B82Y 5/00; C12R 2001/01

(57) ABSTRACT

The present invention relates to methods, compositions, and systems for modulating cellular metabolism in cyanobacteria, particularly F. diplosiphon, through the administration of nanoparticles including zero-valent iron nanoparticles (nZVIs) and titanium dioxide nanoparticles (n-TiO₂), β-lactam antibiotics such as ampicillin, or combinations thereof. The disclosed methods enable controlled alteration of adenosine triphosphate (ATP) concentrations, ATP synthase expression, lactate metabolism, photosynthetic efficiency (Fv/Fm), reactive oxygen species (ROS) levels, thylakoid membrane morphology, and monounsaturated fatty acid (MUFA) production. A combination regimen of ampicillin and nZVIs partially rescues ATP levels otherwise depressed by nZVI treatment alone and enhances MUFA production. Administration of n-TiO₂ at optimal concentrations enhances ATP synthase expression, photosynthetic efficiency, and growth in a strain-specific manner. Applications include sustainable biofuel synthesis and bioproduct manufacturing.

### FIELD OF THE INVENTION

This invention relates to methods, compositions, and systems for modulating cellular energy metabolism, membrane integrity, photosynthetic efficiency, and bioproduct synthesis in cyanobacteria.

### BACKGROUND OF THE INVENTION

The complex interactions between environmental stressors such as nanoparticles and antibiotics have sparked renewed interest in understanding corresponding cellular responses in organisms. Unraveling the interdependent relationship among these external factors reveals the extraordinary capacity of photosynthetic microorganisms, particularly cyanobacteria, to adapt to multifaceted stressors. As versatile microorganisms, cyanobacteria are valuable model organisms for investigating the impact of environmental stressors on metabolic and cellular processes. These organisms, known for their role in renewable energy, have been identified as ideal candidates for understanding microbial lipid synthesis due to their unique metabolic and physiological characteristics. Given their unique photomorphogenic adaptations and photo-regulatory mechanisms, cyanobacteria are capable of optimizing their photosynthetic efficiency in response to varying light conditions, an attribute that directly influences lipid production.

Researchers are increasingly interested in understanding the intracellular processes leading to macromolecule synthesis in cyanobacteria, including adenosine triphosphate (ATP), which provides energy storage, and lactate, an important metabolite involved in the ATP-producing process. As an essential molecule for maintaining cellular vitality, ATP is necessary for enzymatic reactions in lipid synthesis, glycolysis, and the conversion of macromolecules in microorganisms. Thus, the efficient utilization of ATP is crucial for the adequate functioning of these microbial entities. In addition to its role in cellular biosynthesis, ATP is also required for other energy-dependent processes, such as carbon fixation through photosynthesis and nitrogen fixation in cyanobacteria. Various factors needed for ATP synthesis in enzymatic reactions have a substantial impact on the production of macromolecules such as lipids, proteins, and glucose. As a result, a comprehensive understanding of the impact of environmental and cellular factors on cyanobacterial ATP levels is critical for optimizing lipid production, resulting in higher lipid yields.

Lactate, the final product of glycolysis generated by the lactate dehydrogenase (LDH) enzyme, is a crucial molecule in microbial lipid synthesis. Lactate is also known as a biomarker of cellular integrity and membrane status and is influenced by external stressors. With changes in the cyanobacterial cell membrane, lactate dynamically moves out of the cell, resulting in its accumulation in extracellular spaces. Thus, elucidating lactate levels when cultures are exposed to stressors can provide comprehensive insight into membrane-related changes and cellular energy capacity.

Nanoscale zero-valent iron (nZVI) molecules, as iron-rich nanoparticles, provide an essential micronutrient for cyanobacteria and are required for fundamental cellular functions such as photosynthesis and respiration. Furthermore, iron molecules can induce various reactive oxygen species (ROS) via Fenton-type reactions, which position nZVIs in a unique place both through mediation of biosynthesis and in terms of stress-related cellular processes. Ampicillin, as a β-lactam antibiotic, is known to target bacterial cell membranes and enhance their permeability under specific conditions. However, the comprehensive influence of ampicillin on various cyanobacterial species has not yet been fully understood, and different cyanobacterial species exhibit varying levels of susceptibility to this antibiotic.

Titanium dioxide (TiO₂) nanoparticles have garnered significant attention due to their distinct physicochemical properties and wide-ranging applications in environmental remediation, energy production, and biotechnology. Recent studies have highlighted the impact of n-TiO₂ on cyanobacteria, which are key aquatic microorganisms involved in photosynthesis and nitrogen fixation. By modulating stress responses and altering metabolic activities, these nanoparticles can profoundly influence cyanobacterial physiology, thereby altering their ecological functions and interactions within aquatic ecosystems. The interaction of n-TiO₂ with cyanobacterial photosynthetic machinery and cellular antioxidant systems offers a potential pathway to enhance metabolic resilience under environmental stress.

As crucial molecules in cellular signaling, ROS are oxygen-derived molecules that play critical roles in cellular signaling pathways and homeostasis; however, excessive oxidative stress can lead to irreversible damage of cellular components, including proteins, lipids, and DNA. While ROS generation is a natural byproduct of photosynthesis and cellular respiration in cyanobacteria, cellular mechanisms like non-photochemical quenching and increased antioxidant enzyme activity help protect these organisms from oxidative damage.

Despite these advances, there remains a need for methods to controllably and predictably modulate cyanobacterial cellular metabolism—in particular, ATP production, ATP synthase activity, lactate dynamics, photosynthetic efficiency, and fatty acid synthesis—using defined nanoparticle and chemical stressor regimens, both individually and in combination, so as to optimize these organisms for biofuel precursor production, and bioproduct synthesis. There is further a need for understanding how different cyanobacterial strains respond to nanoparticle exposure to enable strain-specific optimization for industrial applications.

### SUMMARY OF THE INVENTION

The present invention addresses the foregoing needs by providing methods, compositions, and systems for the directed modulation of energy and stress metabolism in cyanobacteria, particularly Fremyella diplosiphon (“F. diplosiphon”), through administration of nanoparticles including nZVIs and n-TiO₂, a β-lactam antibiotic, or combinations thereof.

In a first aspect, the invention provides a method for modulating cellular metabolism in a cyanobacterium, comprising: providing a culture of a cyanobacterium of the genus Fremyella; administering to said culture an effective amount of at least one of a β-lactam antibiotic, nZVIs, and n-TiO₂; and cultivating said culture under conditions sufficient to measurably alter at least one of ATP concentration, ATP synthase expression, intracellular lactate concentration, extracellular lactate concentration, photosynthetic efficiency (Fv/Fm), and reactive oxygen species (ROS) levels relative to an untreated control culture.

In a second aspect, the invention provides a method for enhancing ATP production in a cyanobacterium, comprising: providing a culture of F. diplosiphon; administering to said culture an effective amount of a β-lactam antibiotic; and cultivating said culture under conditions sufficient to increase ATP concentration relative to an untreated control, wherein said increase in ATP concentration is associated with a decrease in inter-thylakoidal distance in thylakoid membranes of said cyanobacterium.

In a third aspect, the invention provides a method for inducing a quantifiable oxidative stress response in a cyanobacterium, comprising: contacting a culture of a cyanobacterium with nZVIs at a concentration effective to cause accumulation of said nZVIs proximal to cellular membranes; and cultivating said culture under conditions sufficient to produce a decrease in intracellular ATP concentration, an increase in extracellular lactate concentration, and an increase in mean inter-thylakoidal distance relative to an untreated control.

In a fourth aspect, the invention provides a method for producing biofuel precursors from a cyanobacterium, comprising: administering to a culture of F. diplosiphon strain B481-SD a combination regimen of ampicillin and nZVIs; cultivating said culture under conditions sufficient to induce production of monounsaturated fatty acids (MUFAs) at a level that is at least 2-fold greater than an untreated control; and harvesting said MUFAs.

In a fifth aspect, the invention provides a composition for modulating cyanobacterial metabolism, comprising: a culture medium suitable for growth of a cyanobacterium of the genus Fremyella; and at least one of a β-lactam antibiotic at a concentration of from about 0.1 mg/L to about 5.0 mg/L, nZVIs at a concentration of from about 0.5 mg/L to about 10.0 mg/L, and n-TiO₂ at a concentration of from about 0.5 mg/L to about 128 mg/L.

In a sixth aspect, the invention provides a system for optimizing cyanobacterial bioproduct synthesis, comprising: a bioreactor configured to contain a culture of F. diplosiphon; a light source configured to emit light in the photosynthetically active range; a temperature control apparatus; and reservoirs and a dosing mechanism for administering one or more of a β-lactam antibiotic, nZVIs, and n-TiO₂.

In a seventh aspect, the invention provides a method for enhancing ATP synthase expression in a cyanobacterium, comprising: providing a culture of F. diplosiphon strain B481-SD; administering to said culture an effective amount of n-TiO₂; and cultivating said culture under conditions sufficient to produce a significant increase in ATP synthase protein expression relative to an untreated control culture.

### BRIEF DESCRIPTION OF DRAWINGS

The foregoing summary, as well as the following detailed description of the preferred invention, will be better understood when read in conjunction with the appended drawings. For the purposes of illustrating the invention, there are shown in the drawings embodiments which are presently preferred. It should be understood, however, that the invention is not limited to the precise arrangements and instrumentalities shown. In the drawings:

FIG. 1 depicts a bar graph showing ATP levels in F. diplosiphon B481-SD treated with 0.8 mg/L ampicillin, 3.2 mg/L nZVIs, the combination regimen (0.8 mg/L ampicillin and 3.2 mg/L nZVIs), and untreated control, as measured using a luciferase-based assay. Error bars annotated with different letters denote significant differences (p<0.05), while similar letters indicate no significant differences (p>0.05) among the groups as determined by Tukey's post-hoc test.

FIGS. 2A and 2B depict non-denaturing PAGE and immunoblot analyses of ATP synthase levels in F. diplosiphon strains B481-SD and B481-WT grown in BG-11 medium amended with 0.5, 2.0, and 128 mg/L n-TiO₂, showing (a) immunoblot analysis for detection of ATP synthase protein accumulation and (b) ATP synthase volume in extracted protein lysate.

FIG. 3A depicts growth of F. diplosiphon strain B481-SD treated with nano-titanium dioxide nanoparticles (n-TiO₂) ranging from 0.5 to 128 mg/L. Different letters above the error bars indicate statistical significance among the various treatments (p<0.05).

FIG. 3B depicts growth of F. diplosiphon strain B481-WT treated with nano-titanium dioxide nanoparticles (n-TiO₂) ranging from 0.5 to 128 mg/L. Different letters above the error bars indicate statistical significance among the various treatments (p<0.05).

FIG. 4A depicts photosynthetic efficiency (Fv/Fm) of F. diplosiphon strain B481-SD treated with n-TiO₂ ranging from 0.5 to 128 mg/L. Mean and standard deviations are indicated by error bars.

FIG. 4B depicts photosynthetic efficiency (Fv/Fm) of F. diplosiphon strain B481-WT treated with n-TiO₂ ranging from 0.5 to 128 mg/L. Mean and standard deviations are indicated by error bars.

FIG. 5 depicts intracellular reactive oxygen species (ROS) validated using 2′,7′-dichlorofluorescein (DCF) in F. diplosiphon B481-SD and B481-WT strains grown in BG-11 medium supplemented with 0.5, 2.0, 16, and 128 mg/L n-TiO₂.

FIG. 6 depicts a bar graph showing extracellular lactate levels in B481-SD treated with 0.8 mg/L ampicillin, 3.2 mg/L nZVIs, the combination regimen (0.8 mg/L ampicillin and 3.2 mg/L nZVIs), and untreated control, expressed in terms of fluorescence intensity. Lactate levels were measured using enzymatic assay methods.

FIG. 7 depicts a bar graph showing intracellular lactate levels in B481-SD treated with 0.8 mg/L ampicillin, 3.2 mg/L nZVIs, the combination regimen (0.8 mg/L ampicillin and 3.2 mg/L nZVIs), and untreated control, expressed in terms of fluorescence intensity. Lactate levels were measured using a colorimetric LDH assay

FIG. 8 depicts transmission electron microscopy (TEM) images of inter-thylakoidal distances in F. diplosiphon B481-SD treated with untreated control (A), 0.8 mg/L ampicillin (B), 3.2 mg/L nZVIs (C), and a combination treatment of 0.8 mg/L ampicillin and 3.2 mg/L nZVIs (D). The stack of thylakoid membranes is indicated in red parentheses, and thylakoid membranes are designated “TM.”

### DETAILED DESCRIPTION OF THE INVENTION

Unless otherwise defined, all technical and scientific terms used herein have the same meaning as commonly understood by one of ordinary skill in the art to which this invention belongs. As used herein, the following terms have the meanings ascribed to them below.

The term “nZVIs” or “zero-valent iron nanoparticles” refers to nanoscale particles of elemental iron (Fe⁰) having a particle size generally in the range of about 10 nm to about 100 nm, which function as iron-rich nanoparticles that are essential micronutrients for cyanobacteria and are required for fundamental cellular functions such as photosynthesis and respiration. Iron molecules in nZVIs can induce various reactive oxygen species (ROS) via Fenton-type reactions.

The term “n-TiO₂” or “titanium dioxide nanoparticles” refers to nanoscale particles of titanium dioxide having a nominal particle size generally in the range of about 10 nm to about 100 nm. n-TiO₂ possesses distinct physicochemical properties including photocatalytic, antibacterial, and UV-shielding capabilities, and can modulate stress responses and alter metabolic activities in cyanobacteria.

The term “β-lactam antibiotic” refers to a class of antibiotics containing a β-lactam ring in their molecular structure, including but not limited to ampicillin, which is known to target bacterial cell membranes and enhance their permeability under specific conditions.

The term “combination regimen” refers to the co-administration of two or more agents selected from a β-lactam antibiotic, nZVIs, and n-TiO₂ to a cyanobacterial culture, wherein the agents are present simultaneously in the culture medium during cultivation.

The term “ATP” refers to adenosine triphosphate, an essential molecule for maintaining cellular vitality that is necessary for enzymatic reactions in lipid synthesis, glycolysis, and the conversion of macromolecules in microorganisms.

The term “ATP synthase” refers to the enzyme complex responsible for ATP synthesis, which plays a central role in almost every cellular metabolic process and whose expression can be modulated by nanoparticle exposure.

The term “lactate” refers to the final product of glycolysis generated by the lactate dehydrogenase (LDH) enzyme, which is a crucial molecule in microbial lipid synthesis and a biomarker of cellular integrity and membrane status. Lactate may be measured in both intracellular and extracellular compartments.

The term “inter-thylakoidal distance” refers to the spacing between adjacent thylakoid membranes in a cyanobacterial cell, as measured by transmission electron microscopy, which is relevant to the proton gradient and ATP synthesis capacity of the photosynthetic machinery.

The term “MUFAs” or “monounsaturated fatty acids” refers to fatty acids containing a single double bond in the carbon chain, which are known to reduce oxidative stress and increase cell membrane fluidity.

The term “Fv/Fm” refers to the maximum quantum yield of photosystem II (PSII), calculated as (Fm−Fo)/Fm, where Fm is maximal fluorescence and Fo is minimal fluorescence, and serves as an indicator of photosynthetic efficiency in cyanobacteria.

The term “ROS” or “reactive oxygen species” refers to oxygen-derived molecules that play critical roles in cellular signaling pathways and homeostasis, but whose excessive production can lead to irreversible damage of cellular components including proteins, lipids, and DNA.

As used herein, the term “about” when used in reference to a numerical value means ±10% of the stated value, unless otherwise specified.

### Cyanobacterial Strains and Culture Conditions

The present invention employs cyanobacteria of the genus Fremyella and, in particular, F. diplosiphon, a model cyanobacterium known for its ability to absorb different wavelengths of light. F. diplosiphon serves as a key model organism for studying cyanobacterial adaptation, particularly due to its unique light-regulated complementary chromatic mechanism. Its rapid growth cycle (8-10 days) and ability to acclimate to diverse light conditions make it an ideal system for investigating cellular and metabolic dynamics.

In preferred embodiments, the cyanobacterium is F. diplosiphon strain B481-SD, which has been overexpressed with the sterol desaturase gene for enhanced lipid production (GenBank accession MH329183). In certain embodiments, the cyanobacterium is F. diplosiphon strain B481-WT (wild type, UTEX Algal Repository, Texas). The present invention encompasses the use of either strain, or comparative use of both strains, to elucidate strain-specific responses to nanoparticle and antibiotic treatments.

For nZVI and ampicillin treatment studies, cultures are preferably cultivated in BG-11/HEPES medium under wide-spectrum red light at a wavelength of about 650 nm, at a light fluence rate of about 30 μmol/m²/s. In certain embodiments, the light fluence rate may range from about 10 μmol/m²/s to about 100 μmol/m²/s. More broadly, the monochromatic red light may have a wavelength of from about 620 nm to about 700 nm. Cultivation is carried out with continuous agitation, preferably at about 170 rpm, and at an incubation temperature of about 28° C. More broadly, suitable incubation temperatures range from about 20° C. to about 35° C. Liquid cultures are preferably grown to an optical density of about 0.6 at 750 nm prior to treatment.

For n-TiO₂ treatment studies, F. diplosiphon strains B481-SD and B481-WT are cultivated in liquid BG-11 medium supplemented with 20 mM HEPES. Cultures are grown in 250 mL flasks under constant agitation at 170 rpm and a temperature of 28° C. The light spectrum for photosynthesis is adjusted to 400-700 nm, with an irradiance of 30 μmol/m²/s. Cellular suspensions are initially adjusted to an optical density of 0.1 at 750 nm (OD₇₅₀).

### Treatment Regimens

In practicing the methods of the present invention, cyanobacterial cultures are subjected to one or more of the following treatment conditions: (1) a β-lactam antibiotic administered alone; (2) nZVIs administered alone; (3) n-TiO₂ administered alone; (4) a combination regimen comprising two or more of a-lactam antibiotic, nZVIs, and n-TiO₂; or (5) an untreated control grown in the absence of any treatment.

In a preferred embodiment for nZVI and ampicillin treatments, the β-lactam antibiotic is ampicillin, administered at a concentration of from about 0.1 mg/L to about 5.0 mg/L, and preferably about 0.8 mg/L. The nZVIs are preferably administered at a concentration of from about 0.5 mg/L to about 10.0 mg/L, and preferably about 3.2 mg/L. The nZVIs preferably have a particle size of from about 10 nm to about 100 nm.

In a preferred embodiment for n-TiO₂ treatments, n-TiO₂ is administered at a concentration of from about 0.5 mg/L to about 128 mg/L. In certain preferred embodiments, n-TiO₂ is administered at a concentration of about 2.0 mg/L, which has been identified as an optimal concentration for promoting growth, pigment accumulation, photosynthetic efficiency, and ATP synthase expression in F. diplosiphon B481-SD.

In the combination regimen for nZVI and ampicillin, both agents are co-administered to the culture at the concentrations described above. The entire treatment protocol is preferably conducted in triplicate and repeated at least once for validation. The cultivation period under treatment conditions is preferably from about 5 days to about 20 days, and more preferably about 12 days for nZVI and ampicillin treatments or about 9 to 15 days for n-TiO₂ treatments.

### Modulation of ATP Levels

A cornerstone of the present invention is the discovery that ATP levels and ATP synthase expression in F. diplosiphon can be controllably and differentially modulated depending on the treatment regimen employed. ATP serves as a quintessential energy carrier and informative indicator of shifts in metabolic activity and organismal stress responses, reflecting the dynamic balance between energy production and consumption.

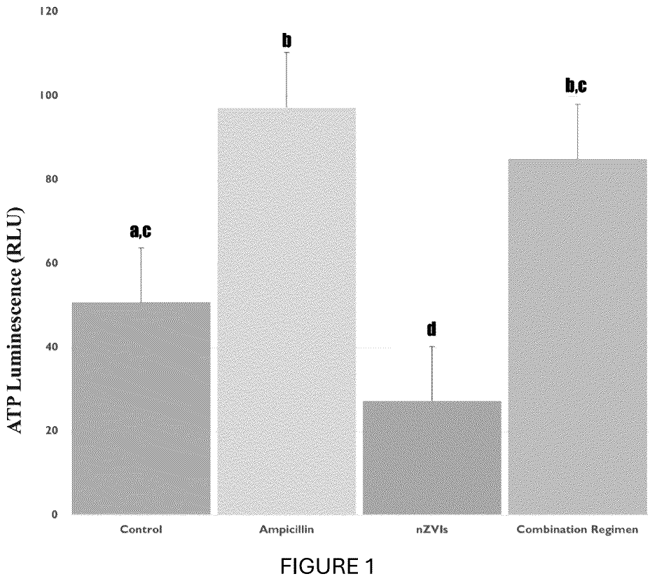

Administration of ampicillin at 0.8 mg/L results in a significant increase (p<0.05) in ATP concentration compared to the untreated control (FIG. 1). Without wishing to be bound by theory, the inventors hypothesize that the enhanced ATP synthesis is an outcome of the organism ramping up cellular activity in an effort to mitigate the potentially deleterious effects of the antibiotic.

In contrast, treatment with 3.2 mg/L nZVIs produces a significant decrease (p<0.05) in ATP levels compared to both the untreated control and the 0.8 mg/L ampicillin treatment (FIG. 1). The stress induced by nZVIs may impair the cellular machinery tasked with ATP synthesis, resulting in a significant decline in ATP yield.

The combination regimen of 0.8 mg/L ampicillin and 3.2 mg/L nZVIs produces an intermediate and particularly advantageous result: ATP levels are significantly elevated (p<0.05) compared to the 3.2 mg/L nZVI treatment alone yet are not significantly different (p>0.05) from the 0.8 mg/L ampicillin treatment and the untreated control (FIG. 1). This suggests an intriguing interplay between the stress responses elicited by the antibiotic and nZVIs. Ampicillin may augment ATP activity in the strain because it is the common treatment in both regimens, reflecting heightened cellular activity and possibly triggering ATP-dependent cellular protection systems.

The substantial differences observed in the various treatments indicate that ampicillin could be linked to its innate ability to impact cell wall synthesis. This effect may stem from increased membrane permeability, facilitating the shift of nZVIs into the intracellular space, therefore mitigating potential detrimental effects that may arise from the accumulation of nZVIs around the cell.

### Modulation of ATP Synthase Expression by n-TiO₂

The present invention further discloses that n-TiO₂ nanoparticles can modulate ATP synthase protein expression in a strain-specific manner in F. diplosiphon. Photosynthetic protein complexes, including photosystem I, cytochrome b6f, and photosystem II, are integral to energy generation and storage in prokaryotes, facilitating essential processes required for cellular metabolism.

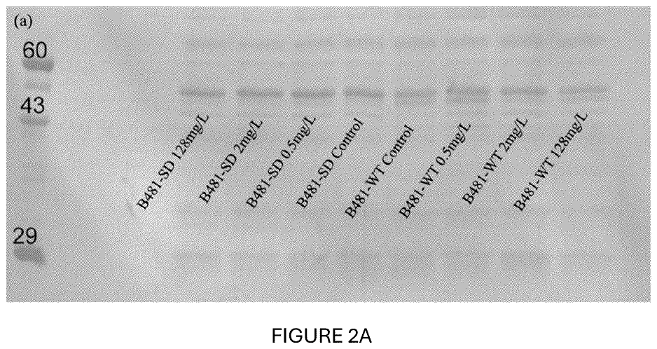

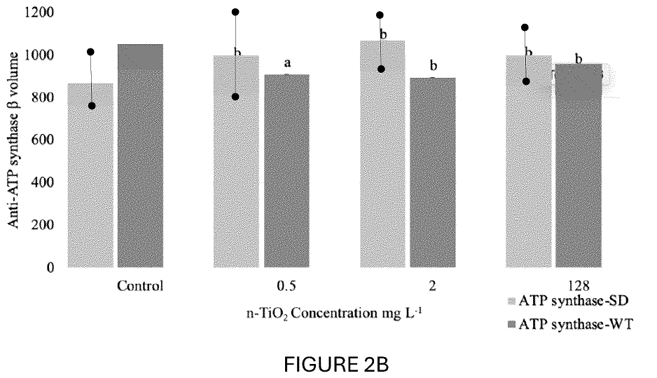

Densitometric analysis of anti-ATP synthase immunoblots (FIG. 2A, with reference bands at 60 kDal, 43 kDal and 29 kDal) indicates a significant increase in ATP synthase expression in B481-SD treated with 0.5, 2.0, and 128 mg/L n-TiO₂ (p<0.05) compared to the untreated control (FIG. 2B). Specifically, significantly higher levels of ATP synthase are observed in B481-SD treated with 2.0 mg/L n-TiO₂ (1061.67±118.98, p<0.01) compared to 0.5 mg/L (997.95±205.20, p<0.01) and 128 mg/L n-TiO₂ (998.54±130.92, p<0.01). Conversely, no significant increase in ATP synthase expression is observed in B481-WT treated with 0.5, 2.0, or 128 mg/L n-TiO₂ compared to the control. Furthermore, ATP synthase levels are significantly higher in the untreated B481-WT (1017.02±113.67, p<0.05) compared to untreated B481-SD (862.39±138.11, p<0.05). These results indicate a cellular adaptation mechanism to nanoparticle-induced stress involving enhanced photosynthetic recovery, attributed to altered metabolic signaling affecting gene expression pathways involved in energy metabolism in B481-SD.

The significantly enhanced ATP synthase activity in n-TiO₂-treated B481-SD could be attributed to the sterol desaturase gene, which is known to increase cyanobacterial membrane fluidity and stability under stress conditions. Enhanced membrane fluidity can facilitate more efficient electron transport across thylakoid membranes, providing protection against nanoparticle-induced oxidative stress and thereby reducing membrane damage.

A previous transcriptome analysis of B481-SD treated with 3.2 mg/L nZVIs revealed a logz fold increase ranging from 1.1× to 1.7× due to the upregulation of photosynthetic genes such as psaI, psaX, psaM, psbK, psbA, and psb30 compared to the untreated control. This suggests a strain-specific regulatory network and stress response mechanisms operative in the B481-SD strain.

### Impact of n-TiO₂ on Growth and Pigment Autofluorescence

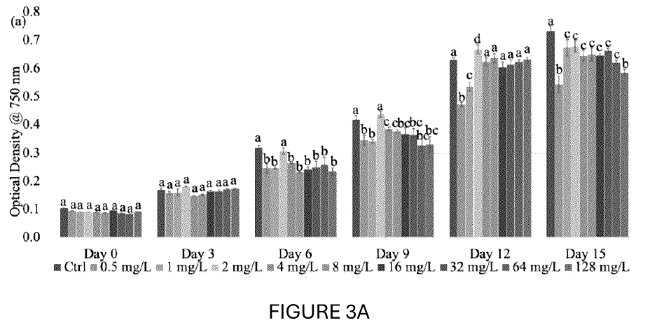

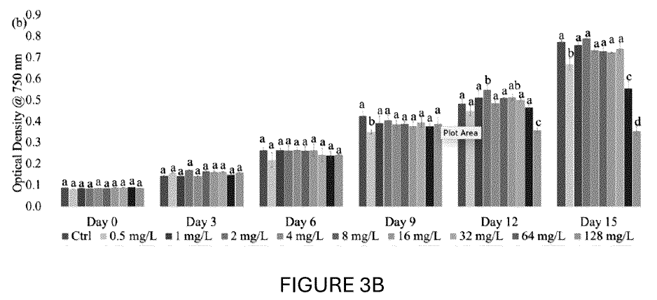

The present invention demonstrates that n-TiO₂ exerts strain-specific effects on the growth and pigment accumulation of F. diplosiphon. Growth measured by optical density reveals significantly higher growth (p<0.05) of B481-SD at 2.0 mg/L n-TiO₂ (0.67±0.01) (FIG. 3A), and of B481-WT at both 2.0 mg/L (0.55±0.01) and 16 mg/L (0.52±0.01) on day 12 (FIG. 3B). Both strains treated with 128 mg/L n-TiO₂ exhibit a significant reduction in growth, indicating potential toxicity that impairs cellular functions.

Measurement of pigment autofluorescence indicates significantly higher phycocyanin (1300±2) and chlorophyll a content (890±5) in B481-SD treated with 2.0 mg/L n-TiO₂ on day 9 compared to the untreated control. However, a significant decrease in phycocyanin content is observed on days 12 and 15 in B481-SD treated with 0.5 to 128 mg/L n-TiO₂. No significant differences (p>0.05) in phycocyanin or chlorophyll content are observed in B481-WT across 0.5 to 128 mg/L n-TiO₂ concentrations. The observed reduction in phycocyanin content in F. diplosiphon B481-SD upon exposure to n-TiO₂ on days 12 and 15 is likely attributed to nanoparticle-induced cytotoxicity, including oxidative stress and disruption of metabolic pathways essential for phycobiliprotein synthesis.

### Impact of n-TiO₂ on Photosynthetic Efficiency (Fv/Fm)

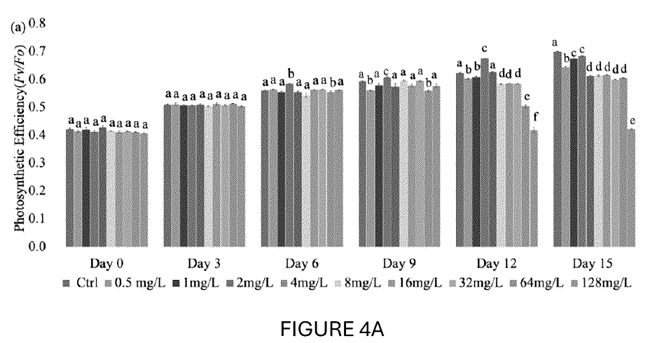

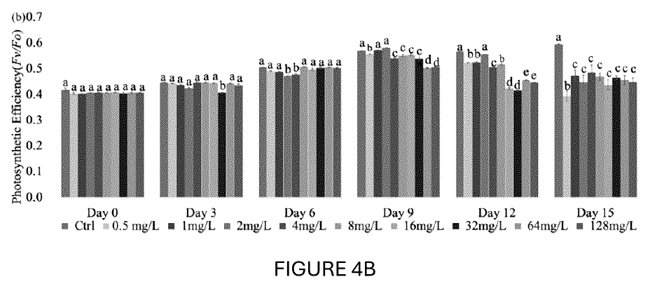

The quantum yield of PSII was measured to assess the photosynthetic efficiency of n-TiO₂-treated F. diplosiphon. Quantification of the Fv/Fm ratio reveals a significant increase (p<0.05) in B481-SD treated with 2.0 mg/L n-TiO₂ on days 6 (0.58±0.01), 9 (0.61±0.03), and 12 (0.68±0.05) compared to the untreated control (FIG. 4A). No significant increase in the Fv/Fm ratio is observed in B481-WT across all n-TiO₂ concentrations ranging from 0.5 to 128 mg/L (FIG. 4B). Both strains exhibit a significant reduction in the Fv/Fm ratio at 128 mg/L n-TiO₂ on days 12and 15, with no signs of recovery.

Without wishing to be bound by theory, the enhanced photosynthetic efficiency in B481-SD suggests possible activation of antioxidant defense systems or structural modifications in the photosynthetic apparatus to enhance light harvesting and energy utilization. This is consistent with findings in Chlamydomonas reinhardtii, where an increased Fv/Fm ratio was reported following exposure to 200 mg/L n-TiO₂, likely as a response to excessive absorbed light energy. The sterol desaturase mutant showed improved growth and higher Fv/Fm values under n-TiO₂ exposure, likely attributable to enhanced ROS scavenging potential.

### Impact of n-TiO₂ on Reactive Oxygen Species Levels

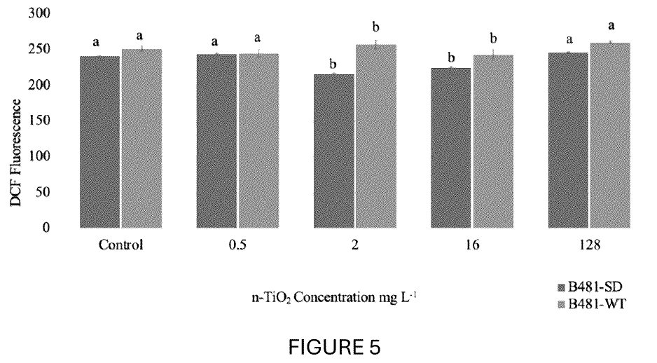

Using the 2′,7′-dichlorodihydrofluorescein diacetate (DCFH-DA) probe, oxidative stress was compared in the untreated control and at varying n-TiO₂ exposure levels (0.5, 2.0, 16, and 128 mg/L) on day 15. While the DCF in B481-SD varied from 220±5 to 245±3 nmol/mL, it ranged from 245±2 to 260±1 nmol/mL in B481-WT. A significant reduction in ROS levels (p>0.01) is observed in B481-SD at 2.0 and 16 mg/L n-TiO₂ compared to the untreated control (FIG. 5). Conversely, a significant increase in ROS levels (260±5) is observed in the B481-WT strain exposed to 2.0 mg/L n-TiO₂.

This strain-specific difference in oxidative stress tolerance may reflect distinct regulatory mechanisms that restrain ROS detoxification. The B481-SD strain's enhanced antioxidant defense responses compared to B481-WT suggest that the sterol desaturase gene modification confers improved resilience to nanoparticle-induced oxidative stress.

### Modulation of Extracellular Lactate Levels

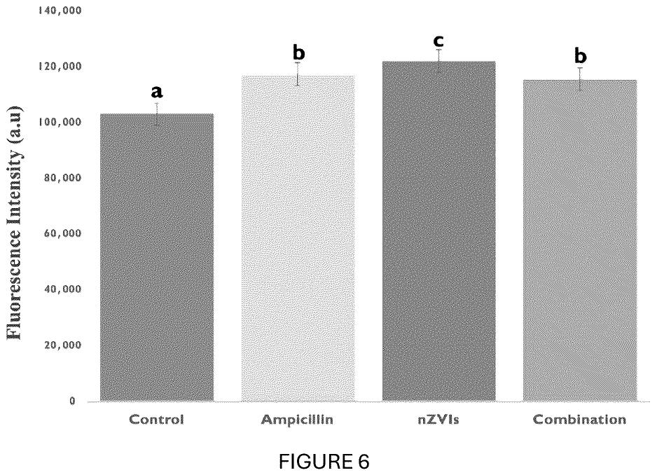

Extracellular lactate levels serve as a valuable marker for cellular stress and membrane integrity under diverse environmental conditions. The present invention demonstrates that all three nZVI/ampicillin treatment regimens—0.8 mg/L ampicillin, 3.2 mg/L nZVIs, and the combination regimen—result in significantly higher (p<0.05) extracellular lactate levels compared to the untreated control (FIG. 6). The increase in cellular stress from external chemicals enhances cellular damage and subsequent lactate release, although the severity of this damage does not reach a level that causes cell death or a decrease in autofluorescence.

Notably, extracellular lactate levels in 3.2 mg/L nZVI-treated cells are significantly higher (p<0.05) than in B481-SD treated with 0.8 mg/L ampicillin or the combination regimen (FIG. 6). Since the release of lactate into the extracellular environment is a well-documented marker of cellular distress, this observation is likely caused by the potent oxidative stress of nZVIs, leading to considerable cellular damage and the release of intracellular components such as lactate. Owing to the small size and high reactivity of nZVIs, these nanoparticles may accumulate rapidly around the cell, impacting the cellular membrane, which subsequently contributes to the observed increase in extracellular lactate.

In response to membrane thinning and enhanced permeability associated with ampicillin, the harmful effects of accumulated nanoparticles around the cell may be mitigated by the leakage of nZVIs into the cells. It is further possible that increased intracellular iron could potentially serve as cofactors for reactions that enhance the synthesis of macromolecules such as lipids and glucose, thereby improving biofuel production efficiency and overall metabolic robustness.

The augmented extracellular lactate levels in 0.8 mg/L ampicillin-treated B481-SD compared to the untreated control may be attributed to the organism's stress response to the antibiotic. By inhibiting the synthesis of cell wall peptidoglycan, ampicillin may induce cellular instability and damage, resulting in the release of LDH into the extracellular space.

The significant increase (p<0.05) in extracellular lactate levels observed in B481-SD treated with ampicillin, nZVIs, and the combination regimen may also be attributed to a shift toward anaerobic metabolism, forcing the cells to rely on glycolysis, where lactate is produced.

Similar ATP and extracellular lactate levels in B481-SD treated with 0.8 mg/L ampicillin and the combination regimen may result from increased membrane permeability. In normal conditions, ATP is contained within cells due to its large size and negative charge, which prevents this essential molecule from leaking into the extracellular space. However, damage to the cell membrane or enhanced membrane permeability can cause ATP, lactate, and similar intracellular products to leak out easily and increase their extracellular levels. In B481-SD treated with 0.8 mg/L ampicillin, 3.2 mg/L nZVIs, the combination regimen, and the untreated control, cell wall thickness was measured at 11.3 nm, 13 nm, 8 nm, and 14 nm, respectively.

### Modulation of Intracellular Lactate Levels

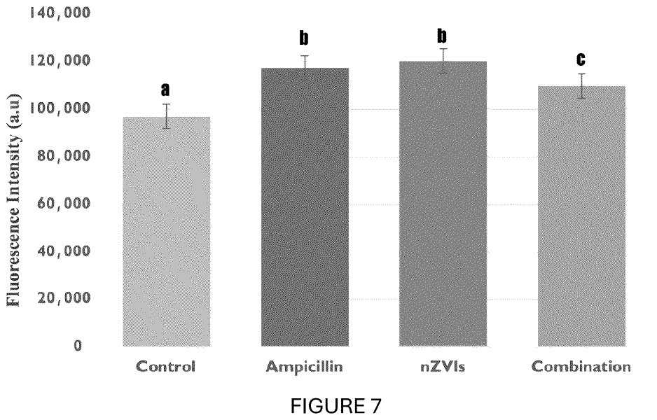

Intracellular lactate levels in B481-SD treated with 0.8 mg/L ampicillin, 3.2 mg/L nZVIs, and the combination regimen are significantly higher compared to the untreated control (FIG. 7). However, the highest intracellular lactate levels are measured in individual treatments of 3.2 mg/L nZVIs and 0.8 mg/L ampicillin, while intracellular lactate under the combination regimen is significantly lower than under either individual treatment.

Without wishing to be bound by theory, increased intracellular lactate levels may indicate a substantial metabolic response in B481-SD treated with nZVIs and ampicillin individually, possibly resulting in increased lactate production or retention within the cells. The significant alteration of metabolic pathways within F. diplosiphon cells in response to antibiotic and nanoparticle stressors is underscored by variations in intracellular lactate levels.

Notably, although the combination regimen includes both chemicals, intracellular lactate levels are significantly lower than under individual treatments. This may be explained by increased production of monounsaturated fatty acids (MUFAs), which are thought to reduce oxidative stress and increase cell membrane fluidity, thereby mitigating detrimental effects and supporting cell survival.

The dichotomy observed between intracellular and extracellular lactate levels may derive from the different roles and regulatory mechanisms of lactate within and outside the cell. Intracellular lactate primarily contributes to metabolic processes such as catalyzing the conversion of pyruvate to lactate during glycolysis and supporting ATP production, while extracellular lactate is primarily a biomarker for cell membrane integrity, as it is released into the extracellular space following damage to the cell membrane or cell death. The observed increase in extracellular lactate levels in B481-SD treated with nZVIs may therefore be attributed to concentration-related changes in the cell wall and membrane.

### Correlation between ATP Dynamics and Cellular Morphology

The present invention further discloses a novel correlation between ATP levels and thylakoid membrane morphology, as revealed by transmission electron microscopy (TEM) analysis. TEM analysis was employed to uncover correlations between cellular ATP levels and distinct morphological changes under each treatment.

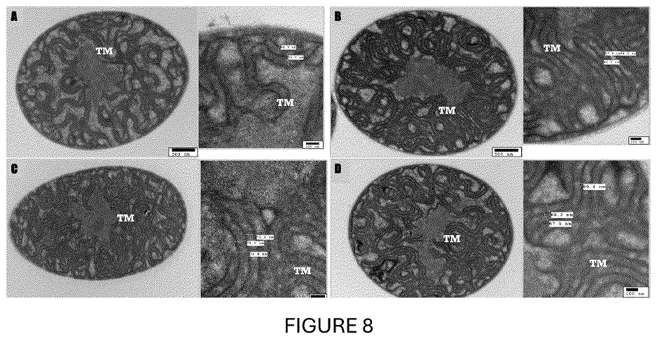

B481-SD treated with 3.2 mg/L nZVIs exhibits a decrease in ATP accompanied by changes in thylakoid membranes and an increase in inter-thylakoidal distance compared to the untreated control and 0.8 mg/L ampicillin. This concurs with the finding that ATP depletion may drive morphological adaptations as part of the cellular stress response. Conversely, increased thylakoid membrane counts in the B481-SD treated with 0.8 mg/L ampicillin correspond to the observed increase in ATP concentrations. ATP changes indicate that disrupted internal organelles might reflect a defensive response to treatments, suggesting that ATP dynamics influence the extent of these morphological responses. The potential for an increased number or volume of thylakoids in the ampicillin, nZVI, and combination regimen compared to the untreated control (FIG. 8) may account for stable ATP levels, as thylakoid membranes are pivotal for ATP synthesis in cyanobacteria.

In the combination regimen and ampicillin treatments, the mean inter-thylakoidal distance among thylakoid membranes was measured at 68.8 nm and 53.86 nm, respectively; however, it was 72 nm and 72.8 nm for the untreated control and nZVIs, respectively (FIG. 8). Therefore, the inter-thylakoidal distance is relevant to ATP synthesis, as shorter distances can result in increased proton gradient, leading to augmented ATP production. Cyanobacterial cells can acclimate to variable environmental changes by modifying thylakoid membranes, which are vital photosynthetic structures, thereby enhancing their resilience.

Disorganized thylakoid membranes and increased intracellular space were observed in TEM studies of nZVI-treated cells, indicating a possible cellular stress response to nZVI exposure. This supports the notion that cellular morphology and energy dynamics are closely intertwined, with changes in ATP levels potentially contributing to morphological adjustments. These findings underline the substantial role of ATP concentration in dictating cellular responses to external stressors, reflected through morphological adaptations.

### Biofuel Precursor and Bioproduct Applications

The methods, compositions, and systems of the present invention find particular application in the production of biofuel precursors and other bioproducts. As disclosed above, the combination regimen of ampicillin and nZVIs enhances MUFA production by 2-3-fold over untreated controls. MUFAs are valuable biofuel precursors and their enhanced production through the combination regimen represents a significant advance in sustainable biofuel synthesis. Moreover, increased intracellular iron from the leakage of nZVIs into cells under conditions of enhanced membrane permeability could potentially serve as cofactors for reactions that enhance the synthesis of macromolecules such as lipids and glucose, thereby improving biofuel production efficiency and overall metabolic robustness.

The enhanced ATP synthase expression in B481-SD treated with n-TiO₂, coupled with improved photosynthetic efficiency and reduced ROS levels, further supports the use of n-TiO₂ as a modulator for optimizing cyanobacterial bioproduct synthesis. Understanding the ability of optimal n-TiO₂ to enhance light capture, improve PSII efficiency, and optimize photosynthetic processes can significantly advance cyanobacteria-based biotechnological applications.

Critical insights into cyanobacterial adaptive mechanisms under nanoparticle and antibiotic stress advance our understanding of environmental stress biology. This knowledge not only facilitates the efficient harnessing of the potential of F. diplosiphon but also paves the way for sustainable and innovative applications across various industries. By understanding the intricacies of F. diplosiphon chemical adaptations, one can unlock its full potential in enhancing value-added bioproducts and biofuel generation for economic viability.

### System Embodiments

In certain embodiments, the present invention provides a system for optimizing cyanobacterial bioproduct synthesis. The system comprises a bioreactor configured to contain a culture of F. diplosiphon, a light source configured to emit light in the photosynthetically active range of from about 400 nm to about 700 nm, and preferably monochromatic red light at a wavelength of from about 620 nm to about 700 nm and more preferably about 650 nm, and a temperature control apparatus configured to maintain a temperature of from about 20° C. to about 35° C. The system further comprises one or more reservoirs containing nanoparticles (nZVIs, n-TiO₂, or both) and optionally a β-lactam antibiotic, and a controller or dosing mechanism configured to administer said agents to the culture in a regimen effective to produce a measurable enhancement in at least one of ATP concentration, ATP synthase expression, MUFA production, and photosynthetic efficiency relative to an untreated control. In preferred embodiments, the system further comprises an agitation mechanism configured to provide continuous shaking of the culture, preferably at about 170 rpm.

### EXAMPLES

### Example 1: Cyanobacterial Strain, Growth Conditions, and Treatments for nZVI and Ampicillin Studies

F. diplosiphon strain B481-SD, which was overexpressed with the sterol desaturase gene for enhanced lipid production (accession MH329183), was used. The organism was cultivated in BG-11/HEPES medium, under wide-spectrum red light (650 nm) at a light fluence rate of 30 μmol/m²/s, using a model LI-190SA quantum sensor (Li-Cor, Lincoln, NE, USA). These conditions were facilitated by an Innova 44R shaker (Eppendorf, Hamburg, Germany), which provided continuous agitation at 170 rpm and an incubation temperature of 28° C. Iron nZVIs were obtained from the Nano Iron Company (Rajhrad, Czechia), and ampicillin, a β-lactam group antibiotic, was obtained from Sigma Aldrich (St. Louis, MO, USA).

B481-SD liquid cultures were grown under the conditions described above to achieve an optical density of 0.6 at 750 nm and subjected to the following treatments: ampicillin at 0.8 mg/L, nZVIs at 3.2 mg/L, and a combined regimen incorporating both ampicillin at 0.8 mg/L and nZVIs at 3.2 mg/L. Control cultures were grown in the absence of any treatment with antibiotics or nZVIs. The entire experiment was conducted in triplicate and repeated once for validation.

### Example 2: ATP Extraction and Measurement

B481-SD cultures grown under the conditions described in Example 1 were harvested on day 12 (0.6 OD₇₅₀) by centrifugation at 13,000 rpm for 3 min. The supernatant was discarded, and pellets were resuspended in 100 μL of 1% ice-cold trichloroacetic acid solution. Samples were vortexed for 30 s and centrifuged at 13,000 rpm for 10 min at 4° C. to collect the supernatant. Trichloroacetic acid solution was neutralized by adding 100 μL of 1 M Tris-Acetate buffer (pH 7.8), and the ATP-containing solution was diluted to 1 mL in deionized water. ATP content was measured using the ATP determination kit (Thermo Fisher Scientific, Molecular Probes Inc., Waltham, MA, USA), according to the manufacturer's instructions. Briefly, 10 μL of the diluted ATP solution was mixed with the ATP detection reagent, and the luminescence signal was measured using a Synergy H1 Multimode microplate reader equipped with Gen5 software version 2.10 (Bio Tek, Santa Clara, CA, USA). ATP concentration was calculated using a standard curve prepared in conjunction with the sample measurement.

A significant increase (p<0.05) in ATP concentration was observed in cultures treated with 0.8 mg/L ampicillin compared to the untreated control (FIG. 1). A significant decrease (p<0.05) in ATP levels was observed in B481-SD treated with 3.2 mg/L nZVIs compared to 0.8 mg/L ampicillin and the combination regimen (FIG. 1); this level was also significantly lower (p<0.05) than the untreated control. ATP levels in the combination regimen of 0.8 mg/L ampicillin and 3.2 mg/L nZVIs were significantly elevated (p<0.05) compared to the 3.2 mg/L nZVI treatment but were not significantly different (p>0.05) from 0.8 mg/L ampicillin and the untreated control (FIG. 1).

### Example 3: Extracellular and Intracellular L-Lactate Quantification

L-Lactate, a metabolic byproduct of glycolysis known for its significant role as an energy provider in cells, was quantified via fluorometric assay. To perform the assay, 1 mL of culture from each treatment was centrifuged at 4000×g for 6 min at 4° C., and the supernatant was preserved at −20° C. Sample analysis was conducted using the L-Lactate Assay kit (Cayman Chemical, Ann Arbor, MI, USA), according to the manufacturer's instructions. The lactate concentrations in the supernatant were corrected using the lactate concentrations in the blanks. Measurements were made on 96-well clear plates (Corning Inc., Corning, NY, USA), using a Synergy H1 Multimode microplate reader.

Extracellular lactate levels in B481-SD grown in 0.8 mg/L ampicillin, 3.2 mg/L nZVIs, and the combination regimen were significantly higher (p<0.05) compared to the untreated control (FIG. 6). Extracellular lactate levels in 3.2 mg/L nZVI-treated cells were significantly higher (p<0.05) than B481-SD treated with 0.8 mg/L ampicillin and the combination regimen (FIG. 6).

Intracellular lactate levels in B481-SD treated with 0.8 mg/L ampicillin, 3.2 mg/L nZVIs, and the combination regimen were significantly higher compared to the untreated control (FIG. 7); however, the highest intracellular lactate levels were measured in individual treatments of 3.2 mg/L nZVIs and 0.8 mg/L ampicillin. Although the combination regimen included both chemicals, intracellular lactate levels were significantly lower than under individual treatments.

### Example 4: Transmission Electron Microscopy

Cultures grown under the conditions described in Example 1 were centrifuged at 4000 rpm for 10 min and fixed with glutaraldehyde to a final concentration of 2.5% for 5 min. The samples were washed thrice with 5 mM HEPES buffer (pH 8.0) and subsequently post-fixed overnight at 4° C. with a 2% solution of potassium permanganate. The cells were rinsed between 8 and 10 times with deionized water and suspended in a 2% solution of SeaKem agarose (FMC Bioproducts, Rockland, ME, USA). Upon solidification, the agarose-embedded samples were sectioned into approximately 2 mm cubes and dehydrated through a series of ethanol baths, incrementally increasing in concentration from 70% to 95% twice for 10 min each, followed by three 20 min immersions in 100% ethanol.

The samples were subsequently prepared for resin infiltration by incubation in propylene oxide for a quarter of an hour. The first stage of resin infiltration was performed in a 1:2 mixture of propylene oxide and Epon for 30 min, followed by a 1:3 mixture for 90 min at room temperature. The agarose cubes were embedded in pure Epon and left to polymerize for 24 h at 40° C., followed by an additional 48 h at 60° C. The polymerized samples were ultrathin sectioned and examined in a Philips Tecnai 10 Transmission Electron Microscope (Phillips, Amsterdam, The Netherlands) at 80 kV. Micrographs were taken with a two-second exposure time on medium format film and subsequently digitized using a transmitted light scanner.

TEM analysis revealed that B481-SD treated with 3.2 mg/L nZVIs exhibited a decrease in ATP accompanied by changes in thylakoid membranes and an increase in inter-thylakoidal distance compared to the untreated control and 0.8 mg/L ampicillin. Increased thylakoid membrane counts were observed in B481-SD treated with 0.8 mg/L ampicillin. In the combination regimen and ampicillin treatments, the mean inter-thylakoidal distance among thylakoid membranes was measured at 68.8 nm and 53.86 nm, respectively; however, it was 72 nm and 72.8 nm for the untreated control and nZVIs, respectively (FIG. 8). Disorganized thylakoid membranes and increased intracellular space were observed in nZVI-treated cells. The minimal inter-thylakoidal distance for photosynthesis (approximately 4 nm based on the size of phycobiliproteins) was maintained in all treatments tested.

### Example 5: Cyanobacterial Strains, Growth Conditions, and n-TiO₂ Treatments

F. diplosiphon strains B481-SD (sterol desaturase-overexpressing strain, accession MH329183) and B481-WT (wild type, UTEX Algal Repository, Texas) were cultivated in liquid BG-11 medium supplemented with 20 mM HEPES. Cultures were grown in 250 mL flasks under constant agitation at 170 rpm and a temperature of 28° C. in an Innova 44R incubator shaker (Eppendorf, Hamburg, Germany). The light spectrum for photosynthesis was adjusted to 400-700 nm, with an irradiance of 30 μmol/m²/s. Titanium dioxide powder (Degussa P-25, 20 nm) was obtained from the Institute of Chemical Education, University of Wisconsin-Madison (Madison, WI, USA).

The effect of n-TiO₂ on the growth of F. diplosiphon B481-SD and B481-WT across a range of concentrations (0.5, 1, 2, 4, 8, 16, 32, 64, and 128 mg/L) was measured using a UV-Vis spectrophotometer (Agilent Technologies, CA, USA). Cellular suspensions were initially adjusted to an optical density of 0.1 at 750 nm. Measurements of OD₇₅₀ were baseline-adjusted using the n-TiO₂-only control for each concentration. The growth was measured at three-day intervals for a period of 15 days. Three biological replicates were maintained for each strain, and the experiment was repeated once.

Significantly higher growth (p<0.05) of B481-SD (FIG. 3A) was observed at 2.0 mg/L n-TiO₂ (0.67±0.01), and of B481-WT (FIG. 3B) at both 2.0 (0.55±0.01) and 16 (0.52±0.01) mg/L on day 12. Both strains exhibited a significant decrease in growth with no recovery at 128 mg/L n-TiO₂ on day 15.

### Example 6: Pigment Autofluorescence in n-TiO₂-Treated Strains

Phycocyanin and chlorophyll a autofluorescence in 0.5 to 128 mg/L n-TiO₂-treated cultures and the untreated control were measured every three days using a BioTek Synergy H1 Microplate Reader (Agilent, United States). A negative control of bare n-TiO₂ was used to account for any potential interference in pigment autofluorescence. Chlorophyll autofluorescence was captured at an excitation wavelength of 420 nm and an emission wavelength of 680 nm, while phycocyanin fluorescence was assessed at an excitation wavelength of 590 nm with emissions at 650 nm.

Pigment autofluorescence indicated significantly higher phycocyanin (1300±2) and chlorophyll a content (890±5) in B481-SD treated with 2.0 mg/L n-TiO₂ on day 9 compared to the untreated control. A significant decrease in phycocyanin content was observed on days 12 and 15 in B481-SD treated with 0.5 to 128 mg/L n-TiO₂. No significant differences (p>0.05) in phycocyanin or chlorophyll content were observed in B481-WT across 0.5 to 128 mg/L n-TiO₂ concentrations.

### Example 7: Photosynthetic Efficiency (Fv/Fm) of n-TiO₂-Treated Strains

Minimal (Fo) and maximal fluorescence yields (Fm) were measured at three-day intervals using a MINI-PAM (Walz, Germany), after a 15-minute dark incubation. The PSII quantum yield (Fv/Fm) was subsequently calculated based on the formula Fv/Fo=(Fm−Fo)/Fo.

The Fv/Fm ratio revealed a significant increase (p<0.05) in B481-SD treated with 2.0 mg/L n-TiO₂ on days 6 (0.58±0.01), 9 (0.61±0.03), and 12 (0.68±0.05) compared to the untreated control (FIG. 4A). No significant increase in the Fv/Fm ratio was observed in B481-WT across all n-TiO₂ concentrations ranging from 0.5 to 128 mg/L (FIG. 4B). Both strains exhibited a significant reduction in the Fv/Fm ratio at 128 mg/L n-TiO₂ on days 12 and 15, with no signs of recovery.

### Example 8: Detection of Intracellular ROS in n-TiO₂-Treated Strains

Using the 2′,7′-dichlorodihydrofluorescein diacetate (DCFH-DA) fluorometric probe (EMD Chemicals, USA), intracellular ROS levels in B481-SD and B481-WT strains were assessed in the untreated control, 0.5, 2.0, 16, and 128 mg/L n-TiO₂, representing a gradient of low, moderate, and extreme cytotoxic effects. Cultures were grown for 15 days under conditions mentioned in Example 5, with three biological replicates for each treatment group, and the experiment was repeated once. For each strain, a negative control containing bare n-TiO₂ was included to account for potential confounding effects due to autofluorescence. A fresh 20 mM DCFDA stock was prepared and 50 μL added to 150 μL of the culture in a 96-well plate. Fluorescence intensity was measured at an excitation of 495 nm and emission of 529 nm using the BioTek microplate reader after incubation in the dark for 45 min at room temperature.

While the DCF in B481-SD varied from 220±5 to 245±3 nmol/mL, it ranged from 245±2 to 260±1 nmol/mL in B481-WT. A significant reduction in ROS levels (p>0.01) was observed in B481-SD at 2.0 and 16 mg/L n-TiO₂ compared to the untreated control (FIG. 5). A significant increase in ROS levels (260±5) was observed in the B481-WT strain exposed to 2.0 mg/L n-TiO₂. The negative control with bare nanoparticles showed no interference with the DCF signal.

### Example 9: ATP Synthase Activity in n-TiO₂-Treated Strains

F. diplosiphon strains B481-SD and B481-WT were grown in BG-11 media amended with 0.5, 2.0, and 128 mg/L n-TiO₂ according to the culture conditions described in Example 5. Untreated cultures devoid of n-TiO₂ served as the control. After a cultivation period of 9 days, cells were harvested, total protein extracted and quantified using the BCA assay. Samples were diluted to a concentration of 2.0 mg/mL in SDS buffer, heated to 95° C. for 10 min, and run on a 10% sodium dodecyl sulfate (SDS) gel.

The blots were rinsed in Tween-20 Tris buffer saline (TTBS), destained in 100% methanol, and blocked for two hours in 5% nonfat dry milk (NFDM) in TTBS. After washing for 3 to 10 min in TTBS, the blots were incubated overnight with the primary antibody (anti-ATP synthase beta subunit [Agrisera, Cat # AS05085] diluted 1:50,000 in 2% NFDM TTBS). Band intensities were quantified above background using Phoretix 1D software (version 11.2). The membranes were treated with secondary anti-Rabbit IgG-HRP antibody (SeraCare, Cat #5220-0337) for 2 h at room temperature. Signal detection was carried out using enhanced chemiluminescence (ECL), and the blots were exposed to Kodak BioMax MR X-ray film (3-minute exposures), and scanned using a laser densitometer (Model PDSI, Molecular Dynamics Inc., Sunnyvale, CA). FIG. 2A, with reference bands at 60 kDal, 43 k Dal and 29 kDal. The scanner was calibrated for linearity using a Neutral Density Filter Set (Melles Griot, Irvine, CA).

Significantly higher levels of ATP synthase were observed in B481-SD treated with 2.0 mg/L n-TiO₂ (1061.67±118.98, p<0.01) compared to 0.5 (997.95±205.20, p<0.01) and 128 mg/L n-TiO₂ (998.54±130.92, p<0.01) (FIG. 2B). No significant increase in ATP synthase expression was observed in B481-WT treated with 0.5, 2.0, or 128 mg/L n-TiO₂ compared to the control. B481-SD exposed to 2.0 mg/L n-TiO₂ exhibited significantly higher ATP synthase activity compared to B481-WT at the same concentration (961.98±122.58, p<0.01).

### Example 10: Statistical Analysis

For nZVI and ampicillin studies, all data were analyzed as the mean +standard deviation of three independent replicates. Statistical analysis was performed using one-way analysis of variance (ANOVA), followed by Tukey's post hoc test, with p<0.05 considered to be statistically significant. SPSS 28.0 (IBM Corporation, Armonk, NY, USA) was used to analyze and plot the data.

For n-TiO₂ studies, statistical analysis was performed using one-way ANOVA with Tukey's post hoc test (p<0.05). The fixed-effect ANOVA model, Yij=μ+αN_i+εij, where Y represents the measured parameter, μ is the overall mean, αN is the n-TiO₂ treatment effect, and εij is the experimental error, was used. Error bars reflect standard deviation (SD).

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A method for modulating cellular metabolism in a cyanobacterium, comprising:
(a) providing a culture of a cyanobacterium of the genus Fremyella;
(b) administering to said culture an effective amount of at least one of: (i) a β-lactam antibiotic, (ii) zero-valent iron nanoparticles (nZVIs), and (iii) titanium dioxide nanoparticles (n-TiO₂); and
(c) cultivating said culture under conditions sufficient to alter at least one of adenosine triphosphate (ATP) concentration, ATP synthase expression, intracellular lactate concentration, extracellular lactate concentration, photosynthetic efficiency (Fv/Fm), and reactive oxygen species (ROS) levels relative to an untreated control culture.

2. The method of claim 1, wherein said cyanobacterium is F. diplosiphon.

3. (canceled)

4. (canceled)

5. The method of claim 1, wherein said β-lactam antibiotic is ampicillin.

6. The method of claim 5, wherein said ampicillin is administered at a concentration of from about 0.1 mg/L to about 5.0 mg/L.

7. (canceled)

8. The method of claim 1, wherein said nZVIs have a particle size of from about 10 nm to about 100 nm.

9. The method of claim 1, wherein said nZVIs are administered at a concentration of from about 0.5 mg/L to about 10.0 mg/L.

10. (canceled)

11. (canceled)

12. The method of claim 1, wherein said n-TiO₂ is administered at a concentration of from about 0.5 mg/L to about 128 mg/L.

13. (canceled)

14. The method of claim 1, wherein step (b) comprises administering both said β-lactam antibiotic and said nZVIs to said culture as a combination regimen.

15. (canceled)

16. (canceled)

17. The method of claim 1, wherein step (c) comprises cultivating said culture under light having a wavelength in the photosynthetically active range of from about 400 nm to about 700 nm.

18. (canceled)

19. The method of claim 1, wherein step (c) comprises cultivating said culture at a temperature of from about 20° C. to about 35° C.

20. The method of claim 1, wherein step (c) comprises cultivating said culture for a period of from about 5 days to about 20 days.

21-39. (canceled)

40. A method for producing biofuel precursors from a cyanobacterium, comprising:
(a) providing a culture of F. diplosiphon strain B481-SD;
(b) administering to said culture a combination regimen comprising: (i) ampicillin at a concentration of from about 0.1 mg/L to about 5.0 mg/L, and (ii) zero-valent iron nanoparticles (nZVIs) at a concentration of from about 0.5 mg/L to about 10.0 mg/L;
(c) cultivating said culture under conditions sufficient to induce production of monounsaturated fatty acids (MUFAs) at a level that is at least 2-fold greater than an untreated control culture; and
(d) harvesting said MUFAs from said culture.

41. The method of claim 40, wherein said ampicillin is administered at a concentration of about 0.8 mg/L and said nZVIs are administered at a concentration of about 3.2 mg/L.

42. The method of claim 40, wherein said combination regimen results in reduced intracellular oxidative stress relative to cultures treated with either ampicillin alone or nZVIs alone.

43. The method of claim 40, wherein said cultivating is performed under monochromatic red light at a wavelength of about 650 nm for a period of about 12 days.

44. A composition for modulating cyanobacterial metabolism, comprising:
(a) a culture medium suitable for growth of a cyanobacterium;
(b) at least one of: (i) a β-lactam antibiotic at a concentration of from about 0.1 mg/L to about 5.0 mg/L, (ii) zero-valent iron nanoparticles (nZVIs) at a concentration of from about 0.5 mg/L to about 10.0 mg/L, and (iii) titanium dioxide nanoparticles (n-TiO₂) at a concentration of from about 0.5 mg/L to about 128 mg/L.

45. The composition of claim wherein said β-lactam antibiotic is ampicillin.

46. The composition of claim 44, wherein said culture medium comprises BG-11 medium buffered with HEPES.

47. The composition of claim 44, wherein said nZVIs have a particle size of from about 10 nm to about 100 nm and said n-TiO₂ has a nominal particle size of from about 10 nm to about 100 nm.

48. The composition of claim 44, further comprising a cyanobacterium of the genus Fremyella cultured therein.

49-52. (canceled)
